(19) United States
(12) Patent Application Publication
(10) Pub. No.: US 2006/0111457 A1
(43) Pub. Date: May 25, 2006

(54) Process for the production of a hydrogen-rich reformate gas by methanol autothermal reforming reaction

(75) Inventors: Chiou-Hwang Lee, Hsinchu (TW); Chien-Liang Hwang, Hsinchu (TW); Jia-Jing Lin, Hsinchu (TW)

Correspondence Address: BACON & THOMAS, PLLC, 625 SLATERS LANE, FOURTH FLOOR, ALEXANDRIA, VA, 22314, US

(73) Assignee: Industrial Technology Research Institute, Hsinchu (TW)

(21) Appl. No.: 11/110,744

(22) Filed: Apr. 21, 2005

(30) Foreign Application Priority Data
Nov. 19, 2004 (TW) 93135735

(51) Int. Cl. C07C 27/26

(52) U.S. Cl. 518/726

(57) ABSTRACT

The present invention provides a catalyst for the methanol autothermal reforming reaction, which includes a mixed oxide of cerium and zirconium as a carrier, and Pt deposited on the carrier. The catalyst of the present invention can catalyze a feed containing methanol, water vapor and air undergoing the autothermal reforming reaction to form a hydrogen-rich reformate gas containing hydrogen, CO and CO₂.

### FIELD OF THE INVENTION

The present invention relates to a catalyst for subjecting methanol to an autothermal reforming reaction (ATR), wherein methanol, air, and steam undergo a reforming reaction to form a hydrogen-rich reformate gas containing hydrogen, CO and CO₂.

### BACKGROUND OF THE INVENTION

A polymer electrolyte fuel cell (PEFC) is highly possible to be applied as a stationary domestic power generation system or in an electric car, and a PEFC system requiring a fuel consisting of a hydrogen-rich gas (concentration of H₂>35%) with a CO concentration lower than 20 ppm. A hydrogen-rich reformate gas formed from a reforming reaction of hydrocarbon contains about 4˜15% of CO, which needs to undergo a water-gas shift (WGS) reaction to reduce the CO content to less than 1%, followed by a preferential oxidation reaction or a methanation reaction and a preferential oxidation reaction in serial in order to reduce the CO concentration to be less than 100 ppm, or even less than 20 ppm. Among fuels, methanol is most suitable to be used as fuel for a mobile PEFC power generation system. Methanol is suitable for the miniaturization of a fuel reformer due to the following properties of methanol: liquid fuel, high energy density per unit volume, portable, free of sulfides, and low reforming temperature. The conventional methanol reformer normally adopts a steam reforming reaction, and seldom adopts an autothermal reforming reaction. An ordinary catalyst for the autothermal reforming reaction of methanol is mainly consisted of Cu—Zn—Al, added with other metals, e.g. Mg, K, Zr, Ca, etc. However, the major drawback of a Cu—Zn—Al catalyst is its poor thermal stability caused by poor thermal stability of Cu per se. When the reaction temperature is high, the activity of Cu will be reduced due to sintering of Cu. In particular, the thermal stability of a Cu—Zn—Al catalyst becomes poorer under ATR reaction conditions.

A PEFC power generation system using methanol as fuel is most suitable for mobile uses. For such an application, the methanol reformer needs to have a compact system volume and a short start-up time. In these respects, the ATR reaction is obviously better than a steam reforming reaction.

Other than a small system volume and a short start-up time, the methanol ATR reformer needs to be able to produce a hydrogen-rich reformate gas with a residual methanol concentration less than 5000 ppm, or even lower, due to the fact that residual methanol will inhibit the activity of the catalyst on the electrodes of the fuel cell stack. In order to reduce the concentration of methanol in a hydrogen-rich reformate gas to be lower than 5000 ppm, the methanol conversion rate for the ATR reaction needs to be higher than 98%. For achieving such an objective, the ATR reaction temperature needs to be increased or the space velocity of the ATR reaction needs to be reduced. The latter is against the demand for reducing the volume of the methanol ATR reformer; and the former (increasing the reaction temperature) is disadvantageous to the existing Cu—Zn—Al catalyst.

The Nissan Automobile Co. and the Mitsubishi Co. have developed a few methanol ATR reaction catalysts containing precious metal. These catalysts mainly consist of Pt—Zn or Pd—Zn, and there are rooms for further improvements, such as the CO concentration being higher than 10% in the hydrogen-rich reformats gas, poor strength of the catalysts, and being vulnerable to pulverize.

### SUMMARY OF THE INVENTION

A primary objective of the present invention is to provide a catalyst for a methanol ATR reaction, which has the properties of high activity and high thermal stability, and thus it can be used to meet the requirements of the methanol ATR reformer: a small reformer volume and a high temperature in the methanol ATR reformer.

A Pt/CeO₂—ZrO₂ catalyst according to the present invention is prepared by mixing a mixed oxide of CeO₂—ZrO₂ as a carrier and a Pt-containing precursor, and drying and calcining the Pt-containing precursor supported on the carrier.

In addition to capable of catalyzing a methanol ATR reaction, the Pt/CeO₂—ZrO₂ catalyst of the present invention has good thermal stability. Meanwhile, a hydrogen-rich reformate gas formed by the methanol ATR reaction catalyzed by the Pt/CeO₂—ZrO₂ catalyst of the present invention has a CO concentration less than 8%, which makes easy for a subsequent water-gas shift reaction to further reduce the CO concentration to less than 1%. When the concentration of CO in a reformate gas is less than 1%, it can be further reduced to less than 100 ppm by methanation or a selective oxidation reaction.

### BRIEF DESCRIPTION OF DRAWINGS

FIG. 1a shows the hydrogen concentration (%) of the reformate gas from a methanol ATR reaction catalyzed by a conventional Cu—Zn—Al catalyst in Control Example 1 vs. the inlet temperature of the reaction gas mixture, wherein the black rhombus dots indicate the first test results, and the hollow rhombus dots indicate the second test results.

FIG. 1b shows the CO concentration (%) of the reformate gas from a methanol ATR reaction catalyzed by a conventional Cu—Zn—Al catalyst in Control Example 1 vs. the inlet temperature of the reaction gas mixture, wherein the black rhombus dots indicate the first test results, and the hollow rhombus dot indicated the second test results.

FIG. 2a shows the hydrogen concentration (%) of the reformate gas from a methanol ATR reaction catalyzed by a commercial MDC-3 catalyst in Control Example 2 vs. the inlet temperature of the reaction gas mixture, wherein the black rhombus dots indicate the first test results, the black square dots indicate the second test results, and the black triangular dots indicate the third test results.

FIG. 2b shows the CO concentration (%) of the reformate gas from a methanol ATR reaction catalyzed by a commercial MDC-3 catalyst in Control Example 2 vs. the inlet temperature of the reaction gas mixture, wherein the black rhombus dots indicate the first test results, the black square dots indicate the second test results, and the black triangular dots indicate the third test results.

FIG. 3a shows the hydrogen concentration (%) of the reformate gas from a methanol ATR reaction catalyzed by the conventional Pd—Zn/Al₂O₃ and Pt—Zn/Al₂O₃ catalysts in Control Examples 3 and 4 vs. the inlet temperature of the reaction gas mixture, wherein the black rhombus dots indicate the results from the Pd—Zn/Al₂O₃ catalyst, and the black square dots indicate the results from the Pt—Zn/Al₂O₃ catalyst.

FIG. 3b shows the CO concentration (%) of the reformate gas from a methanol ATR reaction catalyzed by the conventional Pd—Zn/Al₂O₃ and Pt—Zn/Al₂O₃ catalysts in Control Examples 3 and 4 vs. the inlet temperature of the reaction gas mixture, wherein the black rhombus dots indicate the test results of the Pd—Zn/Al₂O₃ catalyst, and the hollow square dots indicate the test results of the Pt—Zn/Al₂O₃ catalyst.

FIG. 4a shows the hydrogen concentration (%) of the reformate gas from a methanol ATR reaction catalyzed by the Pt/CeO₂—ZrO₂ catalyst in Example 1 of the present invention vs. the inlet temperature of the reaction gas mixture, wherein the black rhombus dots indicate the first test results, the black square dots indicate the second test results, and the black triangular dots indicate the third test results.

FIG. 4b shows the CO concentration (%) of the reformate gas from a methanol ATR reaction catalyzed by the Pt/CeO₂—ZrO₂ catalyst in Example 1 of the present invention vs. the inlet temperature of the reaction gas mixture, wherein the black rhombus dots indicate the first test results, the black square dots indicate the second test results, and the black triangular dots indicate the third test results.

FIG. 5 shows the test results over a long period of time from a methanol ATR reaction catalyzed by a Pt/CeO₂—ZrO₂ catalyst in Example 1 of the present invention at 420° C., wherein the black rhombus dots indicate the hydrogen concentration (%) in the reformate gas, and the black square dots indicate the CO concentration (%) in the reformate gas.

### DETAILED DESCRIPTION OF THE INVENTION

The present invention discloses a method for producing a hydrogen-rich reformate gas by a methanol autothermal reforming reaction, which comprises: undergoing an autothermal reforming reaction of methanol, water and oxygen at 200˜600° C. and in the presence of a catalyst to produce a hydrogen-rich reformate gas comprising hydrogen, CO and CO₂, which is characterized in that said catalyst comprises a mixture of CeO₂—ZrO₂ as a carrier and Pt supported on the mixture oxide.

Preferably, said mixture oxide includes 25˜75% of CeO₂, based on the weight of said mixture oxide.

Preferably, said mixture oxide includes 0.1˜5% of Pt, based on the weight of said mixture oxide.

Preferably, the mole ratio of water to methanol is 1˜3, and the mole ratio of oxygen to methanol is 0.1˜0.8.

A suitable process for preparing the catalyst of the present invention comprises the following steps:

a) impregnating a mixture oxide of CeO₂ and ZrO₂ in an aqueous solution containing Pt ions having an amount so that said mixture oxide is subjected to an incipient wetness impregnation; and

b) heating the resulting impregnated mixture oxide from step a) so that substantially only Pt ions in said aqueous solution are deposited on said mixture oxide.

Preferably, the amount of said aqueous solution in step a) enables said mixture oxide to be impregnated with 0.1-5.0% of Pt ions, based on the weight of said mixture oxide.

Said incipient wetness impregnation comprises measuring the moisture content (ml/g) of said carrier prior to said impregnating, and preparing a suitable volume of an aqueous solution of a Pt metal salt. While under mixing, said aqueous solution is dripped into said carrier in a container, which is immediately absorbed by said carrier. Upon completion of addition of all aqueous solution, said carrier is still in the form of a powder but with slightly wetted surfaces.

Preferably, said heating in step b) comprises drying said incipient wetness impregnated mixture oxide at 100-150° C., and calcining said dried mixture oxide at 400-600° C., preferably at 450-550° C.

Another suitable process for preparing the catalyst of the present invention comprises the following steps:

A) immersing a mixture of CeO₂—ZrO₂ in an aqueous solution containing Pt ions;

B) adding a precipitation agent into said aqueous solution for precipitation of Pt ions on said mixture oxide in a metal form;

C) separating the mixture obtained in step B) by filtration in order to obtain a mixture oxide precipitated with Pt metal; and

D) heating said mixture oxide precipitated with Pt metal.

Preferably, the precipitation agent in step B) is selected from hydrazine or formaldehyde.

Preferably, the amount of said aqueous solution in step A) enables said mixture oxide to be precipitated with 0.1-5.0% of Pt, based on the weight of said mixture oxide.

Preferably, said heating in step D) comprises drying said mixture oxide precipitated with Pt metal at 100-150° C., and calcining said dried mixture oxide at 400-600° C., more preferably at 450-550° C.

The present invention is further elaborated by the following examples, which are for illustrative purposes only and not for limiting the scope of the present invention.

### CONTROL EXAMPLE 1

151.02 g of Cu(NO₃)₂.3H₂O, 214.31 g of Al(NO₃)₃.9H₂O and 71.0 g of Zn(NO₃)₂.6H₂O were weighed and dissolved in 3000 ml of deionized water. The resulting mixture was mixed at room temperature, and 28% ammonia water was added dropwise to a pH value of 7.5. After mixing at room temperature for 2 hours, the resulting mixture was filtered, water washed, dried at 120° C. (for 24 hours), and calcined at 500° C. for 5 hours, thereby obtaining a Cu/Al₂O₃—ZnO catalyst with a weight composition of CuO:ZnO:Al₂O₃=39:15.3:45.7. Said catalyst was added with 1 wt % of silica as a binder, and the resulting mixture was compressed to form tablets, which were then broken into pellets of 16˜20 mesh.

### CONTROL EXAMPLE 2

A MDC-3 commercial catalyst purchased from the Sud-Chemi Co. (Germany) was ground to form a granular catalyst of 16˜20 mesh, wherein the main composition of the MDC-3 catalyst was CuO:ZnO:Al₂O₃=40˜44:44˜50:7˜13 (weight ratio).

### CONTROL EXAMPLE 3

5.3 g of Zn(NO₃)₂.6H₂O (95%) and palladium nitrate containing 1.8 g of palladium were dissolved in 66 ml of deionized water. To the resulting solution 60 g of alumina powder was slowly added, and the resulting mixture was dried at 120° C. for 24 hours, and calcined at 300° C. for 5 hours. The calcination product was mixed with 1 wt % of silica as a binder, which was compressed to form tablets, and then ground to form a granular Pd—Zn/Al₂O₃ catalyst of 16˜20 mesh.

### CONTROL EXAMPLE 4

2.74 g of Zn(NO₃)₂.6H₂O (95%) and platinum nitrate containing 1.8 g of Platinum were dissolved in 66 ml of deionized water. To the resulting solution 60 g of alumina powder was slowly added, and the resulting mixture was dried at 120° C. for 24 hours, and calcined at 300° C. for 3 hours. The calcination product was mixed with 1 wt % of silica as a binder, which was compressed to form tablets, and then ground to form a granular Pt—Zn/Al₂O₃ catalyst of 16˜20 mesh.

### EXAMPLE 1

A mixture oxide powder of CeO₂ and ZrO₂ containing 75 wt % of CeO₂ was used as a carrier. An incipient wetness impregnation method was used to impregnate a solution containing platinum nitrate on the carrier. Said incipient wetness impregnation method was carried out by dissolving platinum nitrate in deionized water to form 100 ml of aqueous solution containing 5 g of platinum, slowly adding said aqueous solution on 250 g of the carrier, drying the incipient wetness impregnated carrier at 120° C. for 24 hours, calcining the dried carrier at 500° C. for 2 hours to form a Pt/CeO₂—ZrO₂ catalyst containing 2 wt % of platinum. Said catalyst was added with 1 wt % of silica as a binder, and the resulting mixture was compressed to form tablets, which were then broken into pellets of 16˜20 mesh.

A conventional fixed-bed reaction system was used to test the activity of the catalysts in the methanol ATR reaction. The above-mentioned granular catalyst (16˜20 mesh) was disposed in a quartz reaction tube with an inside diameter of 2.2 cm. An electric heating furnace was used to control the inlet temperature of the reaction gas mixture. The reaction gas mixture had a mole ratio of H₂O/methanol of 1.3 or 1.8, and a mole ratio of oxygen/methanol of 0.25. The flow rate of the reaction gas mixture was 4 L/min, and the volume of the catalyst was 6.3 ml.

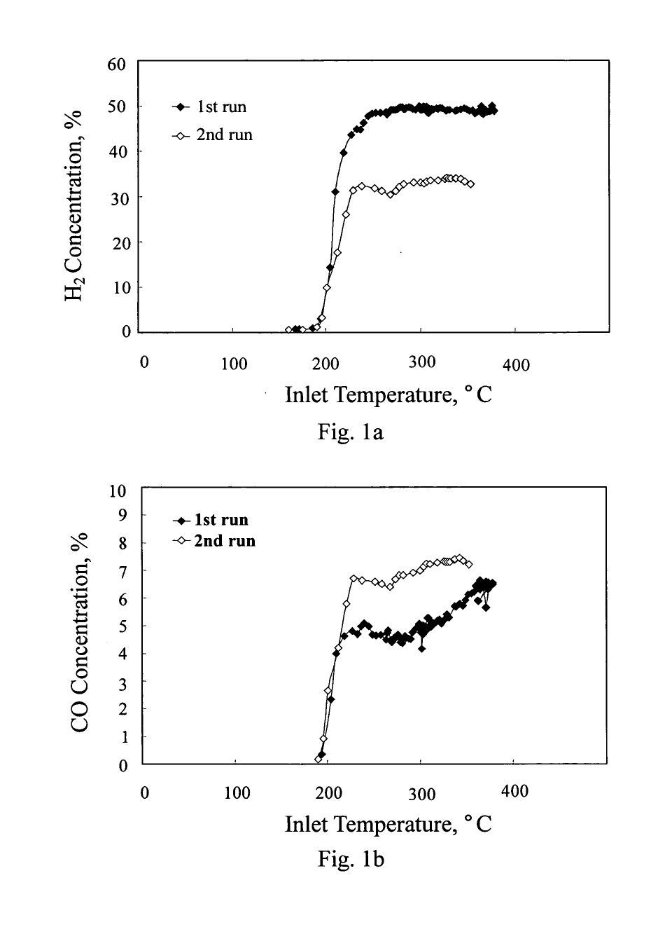

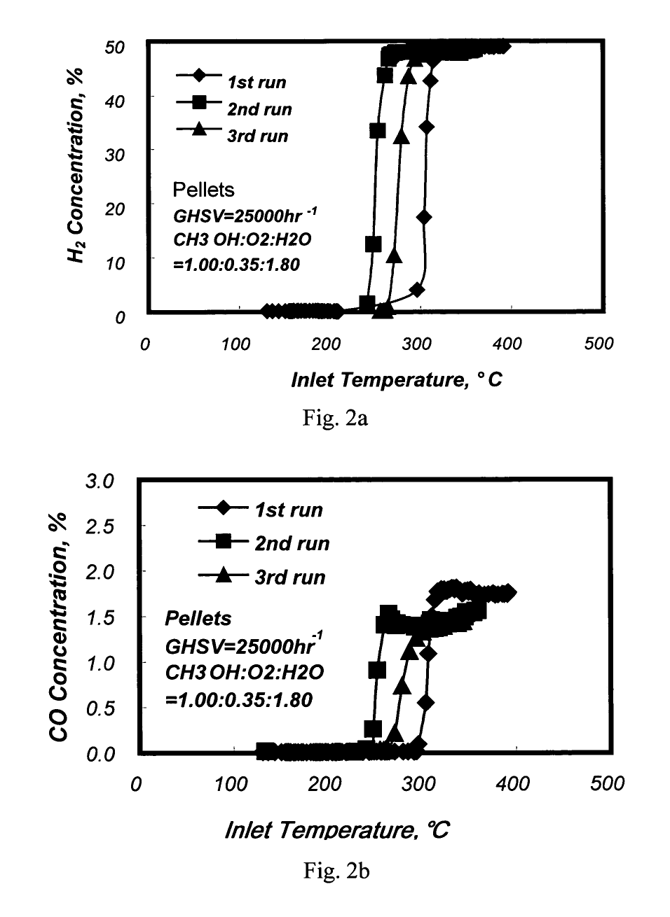

FIGS. 1˜5 show the test results of the catalysts prepared in Control Examples 1-4 and Example 1 in the methanol ATR reaction. The experimental results in FIGS. 1a and 1b show that the conversion ratio of methanol for the conventional Cu—Zn—Al catalyst (Control Example 1) decreases rapidly in the methanol ATR reaction. This indicates that such a catalyst has a poor thermal resistance. For the MDC-3 commercial catalyst (Control Example 2), the methanol ATR reaction process was repeated, and this catalyst is obviously more stable than the Cu—Zn—Al catalyst in Control Example 1. However, the combined concentration of CO and hydrogen in the reformate gas generated by this catalyst still shows a trend of decrease. Furthermore, under 24 hours of continuous operation, this catalyst pulverizes seriously, and only about 50 vol % of the catalyst left at the end of the 24-hr operation.

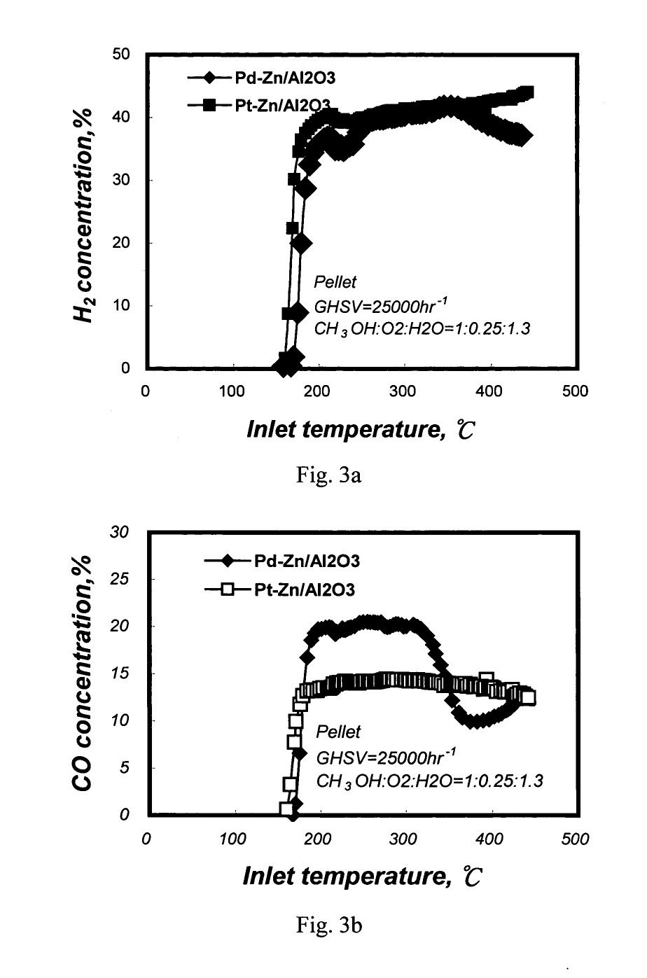

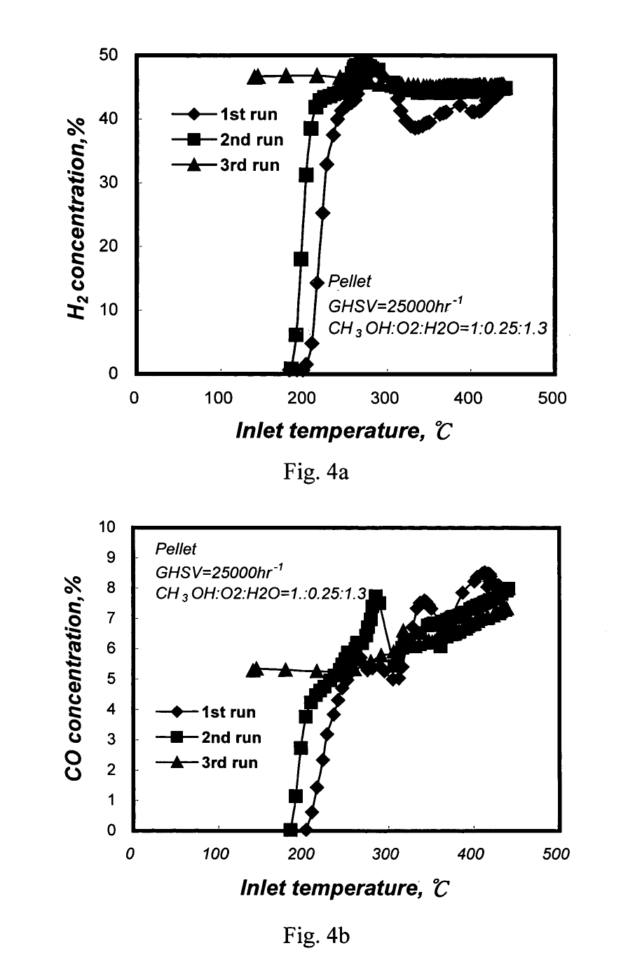

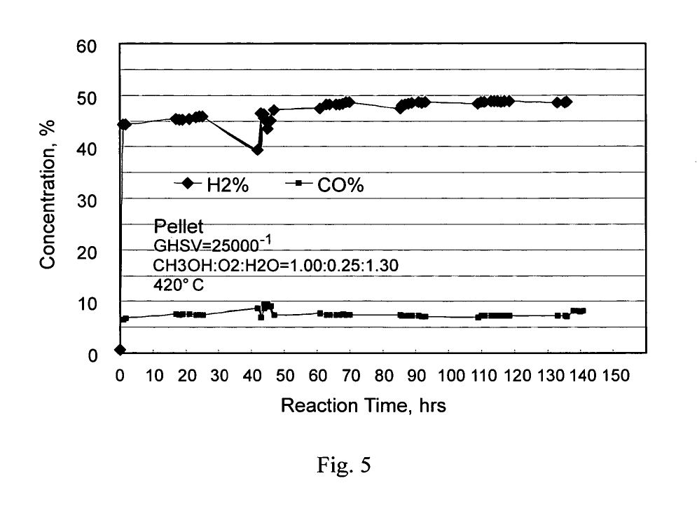

Even though the Pt—Zn/Al₂O₃ and Pd—Zn/Al₂O₃ catalysts of the Control Examples 3 and 4 show a high activity in the methanol ATR reaction (FIG. 3a), the CO concentration in the hydrogen-rich reformate gas generated by these catalysts is obviously higher (>10%) (FIG. 3b). This is disadvantageous to the subsequent reduction of CO concentration in the hydrogen-rich reformate gas. Only the Pt/CeO₂—ZrO₂ synthesized in Example 1 shows a higher stability in the repeated methanol ATR reaction process, as shown in FIGS. 4a and 4b. During an endurance test on the catalyst prepared in Example 1 of the present invention in the above-mentioned methanol ATR reaction at 420° C., the hydrogen-rich reformate gas produced has a stable H₂ concentration and a stable CO concentration (FIG. 5). This indicates that the thermal stability of the Pt/CeO₂—ZrO₂ of the present invention is significantly better than that of the conventional Cu—Zn—Al catalyst, and the CO concentration in the hydrogen-rich reformate gas generated is lower than 10%.

The present invention had been described in the above. Any person skilled in the art still could provide various variations and modifications to the present invention without departure from the scope of the present invention, which is defined in the following claims.

1. A method for producing a hydrogen-rich reformate gas by a methanol autothermal reforming reaction, which comprises: performing an autothermal reforming reaction of methanol, water and oxygen at 200˜600° C. and in the presence of a catalyst, producing a hydrogen-rich reformate gas comprising hydrogen, CO and CO₂, wherein the improvement comprises that said catalyst comprises a mixture oxide of CeO₂ and ZrO₂ as a carrier, and Pt deposited on said mixture oxide.

2. The method as claimed in claim 1, wherein said mixture oxide comprises 25˜75% of CeO₂, based on the weight of said mixture oxide.

3. The method as claimed in claim 1, wherein said catalyst comprises 0.1˜5% of Pt, based on the weight of said mixture oxide.

4. The method as claimed in claim 1, wherein said water and said methanol is in a mole ratio of water to methanol is 1˜3, and said oxygen and said methanol is in a mole ratio of oxygen to methanol is 0.1˜0.8.

5. The method as claimed in claim 1, wherein said catalyst is prepared by a process comprising the following steps:
a) impregnating a mixture oxide of CeO₂ and ZrO₂ in an aqueous solution containing Pt ions having an amount so that said mixture oxide is subjected to an incipient wetness impregnation; and
b) heating the resulting impregnated mixture oxide from step a) so that substantially only Pt ions in said aqueous solution are deposited on said mixture oxide.

6. The method as claimed in claim 5, wherein the amount of said aqueous solution in step a) enables said mixture oxide to receive an incipient wetness impregnation of 0.1-5.0% of Pt ions, based on the weight of said mixture oxide.

7. The method as claimed in claim 5, wherein said heating in step b) comprises drying said incipient wetness impregnated mixture oxide at 100-150° C., and calcining said dried mixture oxide at 400-600° C.

8. The method as claimed in claim 7, wherein said calcining is carried out at 450-550° C.
